(19) United States
(12) Patent Application Publication
(10) Pub. No.: US 2023/0201348 A1
(43) Pub. Date: Jun. 29, 2023

(54) NITRIC OXIDE HYDROGEL FOR PROMOTING TUMOR VASCULAR NORMALIZATION AND RADIOSENSITIZATION AND PREPARATION METHOD THEREOF

(71) Applicant: Nankai University, Tianjin (CN)

(72) Inventors: Jie Gao, Tianjin (CN); Ying Zhang, Tianjin (CN)

(21) Appl. No.: 18/067,991

(22) Filed: Dec. 19, 2022

(30) Foreign Application Priority Data
Dec. 24, 2021 (CN) 202111604730.9

(51) Int. Cl. A61K 41/00; A61P 35/00; A61K 9/06; A61K 33/00

(52) U.S. Cl. CPC A61K 41/0038; A61P 35/00; A61K 9/06; A61K 33/00

(57) ABSTRACT

The present disclosure provides a nitric oxide hydrogel for promoting tumor vascular normalization and radiosensitization and a preparation method thereof. The hydrogel includes a gel-forming polypeptide for forming a hydrogel and a β-galactose-protected NO donor molecule, where the gel-forming polypeptide and the β-galactose-protected NO donor molecule are covalently linked. In the present disclosure, the preparation method has a low synthesis cost, and adopts daily essential amino acid of the human body as raw materials, showing desirable biocompatibility. The hydrogel acts as a NO reservoir for continuous NO delivery on demand, which significantly solves the problem of a short half-life of NO molecules. Most importantly, the hydrogel releases NO only under the catalysis of β-galactosidase (β-Gal), with a release amount precisely controlled by an enzyme concentration.

### CROSS REFERENCE TO RELATED APPLICATION

This patent application claims the benefit and priority of Chinese Patent Application No. 202111604730.9, filed with the China National Intellectual Property Administration on Dec. 24, 2021, the disclosure of which is incorporated by reference herein in its entirety as part of the present application.

### TECHNICAL FIELD

The present disclosure belongs to the field of tumors, and in particular relates to a nitric oxide hydrogel for promoting tumor vascular normalization and radiosensitization and a preparation method thereof.

### BACKGROUND

In cancer radiotherapy, the hypoxic environment at tumor sites generally leads to the radioresistance of solid tumors. Nitric oxide (NO) is an important gas molecule associated with blood vessels and having multiple biological functions, showing multiple effects on hypoxic tumors. NO is a potent hypoxic cellular radiosensitizer. NO has a similar electron affinity to oxygen and can bind to free radicals that damage DNA to fix the damages. In addition to this direct effect, NO can also reduce tumor hypoxia in an indirect manner by normalizing vasculature in the tumor microenvironment. Normalization of tumor vasculature can enhance tumor blood perfusion and ultimately increase the oxygen supply to tumor cells, making tumors sensitive to radiotherapy. Accordingly, delivering NO to tumors becomes a promising approach to reverse tumor resistance to radiotherapy.

Due to the risks in vivo with the direct use of NO gas, some exogenous NO donor materials have been developed as radiosensitizers in academia, including materials that spontaneously release NO molecules directly in solutions, and some materials that release NO under stimulations such as pH, temperature, and light illumination. Despite these successes in NO delivery, none of these studies achieved clinical application. A more precise delivery material design is still required because of the complex functions and mechanisms of NO. Studies have shown that different NO dosages can lead to completely different or even opposite biological functions. In addition, the timing is also important for NO release therapy. In the case of radiosensitization, the normalization of tumor blood vessels to reduce hypoxia requires continuous stimulation by a certain concentration of NO. However, a large amount of NO is required to produce maximum DNA damage and cytotoxicity to cancer cells after radiation exposure, which may also cause damages to normal cells. The NO-releasing materials currently developed can release a large amount of NO in a short period of time, but cannot continuously release NO at a low dosage. At present, there is still a lack of a delivery material that can precisely control the quantity and duration of the NO sustained release for radiosensitization.

### SUMMARY

In view of this, the present disclosure aims to provide a nitric oxide hydrogel for promoting tumor vascular normalization and radiosensitization and a preparation method thereof. The present disclosure may solve the problem that a therapeutic effect of the NO donor that spontaneously releases NO is limited by an extremely short half-life of NO, and may also solve the problem that the current NO donor lacks continuous controlled release in quantity and duration.

To achieve the above objective, the present disclosure adopts the following technical solutions.

The present disclosure provides a nitric oxide hydrogel for promoting tumor vascular normalization and radiosensitization, including a gel-forming polypeptide for forming a hydrogel and a β-galactose-protected NO donor molecule, where the gel-forming polypeptide and the β-galactose-protected NO donor molecule are covalently linked.

In the present disclosure, a supramolecular hydrogel is designed as a NO reservoir for continuous NO delivery on demand. The supramolecular hydrogel is self-assembled from galactose-protected NO donors and release NO only under the catalysis of β-galactosidase (β-Gal). The mechanism is: the β-Gal removes a galactose group of SupraNO, liberates the NO donor, and then releases two molecules of NO. The amount released can be precisely controlled by an enzyme concentration.

The design is based on the following aspects: β-Gal is reported to be overexpressed by various cancer cells including human ovarian cancer and melanoma. Therefore, following intratumoral injection of the hydrogel, the β-Gal in a tumor environment continuously triggers NO release, providing local sustained release of NO at a low dosage. Meanwhile, immediate intravenous injection of a large amount of the β-Gal after radiotherapy is capable of releasing a large amount of NO. In conclusion, the hydrogel is designed to control the position, concentration, and duration of NO exposure in vivo, while having the dual functions of promoting vascular normalization and radiosensitization in hypoxic tumors.

Preferably, the gel-forming polypeptide is a polypeptide including amino acid sequences GFFY and FFG, or an active fragment, an analog, or a derivative of the polypeptide;

preferably, the gel-forming polypeptide has a general formula being one of R-GFFY, R-GFFYG, R-GFFYGG, R-GFFYGGG, R-FF, R-FFG, R-FFGG, and R-FFGGG, and R is selected from the group consisting of H, acetic acid (Ac-), naphthylacetic acid (Nap-), and 9-fluorenylmethoxycarbonyl (Fmoc-); and

more preferably, the gel-forming polypeptide further includes alkynyl located at a C-terminus of the polypeptide or on one of the amino acid side chains of the polypeptide.

The alkynyl can react with an azide group of the NO donor molecule, including terminal alkynes and non-terminal alkynes; alkynes can only be located at a C-terminus of the polypeptide or on an amino acid side chain of the polypeptide, but cannot be located at other positions in a polypeptide backbone.

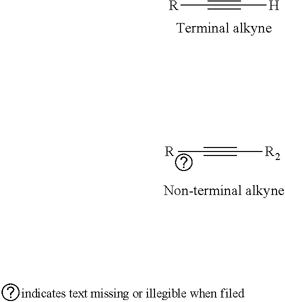

Preferably, in the GFFY, each FFY has an L-configuration or a D-configuration; and in the FFG, each FF has the L-configuration or the D-configuration.

Preferably, the β-galactose-protected NO donor molecule has a structural formula as follows:

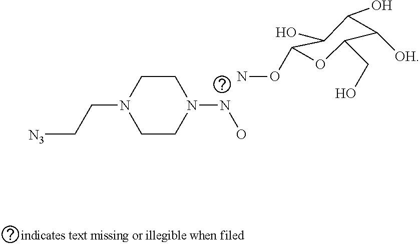

Preferably, the hydrogel releases NO under the catalysis of β-galactosidase (β-Gal).

Preferably, after intratumoral injection of the hydrogel, the β-Gal in a tumor environment continuously triggers NO release, providing local sustained release of NO at a low dosage; and

immediate intravenous injection of the β-Gal after radiotherapy is capable of releasing a large amount of NO.

The present disclosure further provides a preparation method of the hydrogel, including the following steps:

S1: synthesizing the gel-forming polypeptide by polypeptide solid-phase synthesis; and

S2: linking the β-galactose-protected NO donor molecule to the gel-forming polypeptide by Click chemistry.

The present disclosure further provides use of the hydrogel in preparation of a drug for radiotherapy of cancer.

Compared with the prior art, the nitric oxide hydrogel of the present disclosure has the following beneficial effects:

(1) the hydrogel has a low synthesis cost, and adopts daily essential amino acid of the human body as raw materials, showing desirable biocompatibility;

(2) the hydrogel acts as a NO reservoir for continuous NO delivery on demand, which significantly solves the problem of a short half-life of NO molecules; and

(3) the supermolecular hydrogel releases NO only under the catalysis of β-galactosidase (β-Gal), with a release amount precisely controlled by an enzyme concentration.

Compared with the prior art, the preparation method has the same advantages as those of the hydrogel, which are not repeated here.

### BRIEF DESCRIPTION OF THE DRAWINGS

As a part of the present disclosure, the accompanying drawings of the specification provide further understanding of the present disclosure. The schematic embodiments of the present disclosure and description thereof are intended to explain the present disclosure and are not intended to constitute an improper limitation to the present disclosure. In the accompanying drawings:

FIG. 1 shows an optical photograph of the hydrogel;

FIG. 2 shows an electron microscope image of nanofibers inside the NO hydrogel;

FIG. 3 shows a chemical structural formula of a gel-forming molecule 3, and a principle diagram of enzyme-catalyzed release of NO;

FIG. 4 shows determination of enzymatically-controlled release of NO by the NO hydrogel (1 mg/ml) in the plasma of mice;

FIG. 5 shows determination of enzymatically-controlled release of NO by the NO hydrogel (5 mg/ml) in the PBS;

FIG. 6 shows a statistical diagram of colony formation (survival rate) after radiotherapy of B16 cells treated with different groups under hypoxia;

FIG. 7 shows a statistical diagram of a tumor volume of mouse melanoma after combined treatment of the NO hydrogel with radiotherapy;

FIG. 8 shows a tumor photo of mouse melanoma after combined treatment of the NO hydrogel with radiotherapy; and

FIG. 9 shows a statistical result of the NO hydrogel in promoting vascular normalization (perivascular cell coverage ratio) at tumor sites.

### DETAILED DESCRIPTION OF THE EMBODIMENTS

Unless otherwise defined, the technical and scientific terms used in the following examples have the same meanings as commonly understood by those skilled in the art to which the present disclosure belongs. Unless otherwise specified, in the following examples, the test reagents used are all conventional biochemical reagents, and the test methods are all conventional methods.

The present disclosure will be described in detail below with reference to the accompanying drawings and the examples.

### Example 1

(1) Synthesis of a Compound 1

The method included the following specific steps:

1) 0.5 mmol of a 2-cl-Trt resin was placed in a solid-phase synthesizer, 10 mL of anhydrous dichloromethane (DCM) was added, and a mixture was shaken on a shaking table for 5 min to make the 2-Cl-Trt resin fully swollen;

2) the DCM was removed using a rubber suction bulb from the solid-phase synthesizer loaded with the 2-Cl-Trt resin;

3) 0.75 mmol of an Fmoc-protected amino acid was dissolved in 10 mL of anhydrous DCM, 0.75 mmol of DIEPA was added, a mixture was transferred to the solid-phase synthesizer, and 0.75 mmol of the DIEPA was added, and a reaction was conducted at a room temperature for 1 h;

4) closure: the reaction solution in the solid-phase synthesizer was removed with the rubber suction bulb, and then washed with 10 mL of the anhydrous DCM at 1 min each time for a total of 5 times; 20 mL of a prepared solution containing anhydrous DCM, DIEPA, and methanol in a volume ratio of 17:1:2 was added, and a reaction was conducted at a room temperature for 10 min;

5) the reaction solution in the solid-phase synthesizer was removed using a rubber suction bulb, and the synthesizer was washed with 10 mL of the anhydrous DCM at 1 min each time for a total of 5 times, and washed with 10 mL of dimethylformamide (DMF) at 1 min each time for a total of 5 times; the synthesizer was added with 10 mL of DMF containing 20% piperidine by volume to conduct a reaction for 25 min, and 10 mL of the DMF containing 20% piperidine by volume was added to conduct a reaction for 5 min, and then washed with 10 mL of the DMF at 1 min each time for a total of 5 times, to prepare for a next reaction;

6) 1 mmol of a second Fmoc-protected amino acid, 1.5 mmol of HBTU, 2 mmol of DIEPA, and 10 ml of DMF were mixed, a prepared solution was added to the solid-phase synthesizer to conduct a reaction for 2 h;

7) steps 5) and 6) were repeated, the required amino acid or end-capping group (2-naphthylacetic acid) was added successively, and then washed 5 times with DMF, washed 5 times with DCM, to prepare for a next step;

8) 10 mL of a solution consisting of 95% of TFA, 2.5% of TIS, and 2.5% of H₂O by volume was added to the solid-phase synthesizer, and reacted for half an hour (or the TFA and the DCM at a volume ratio of 1:99 were prepared into a TFA solution with a volume percent concentration of 10%, and the TFA solution was added to the solid-phase synthesizer, at each 3 mL for ten times in total with a reaction of 1 min in each time); a product was cut from the 2-cl-Trt resin, concentrated in vacuum, and the solvent was removed to obtain a crude product, which was then separated and purified by HPLC to obtain Nap-GFFYG; and

9) 1.0 mmol (651.7 mg) of the Nap-GFFYG, 1.1 mmol (416.9 mg) of the HBTU, and 2.2 mmol (284.4 mg) of a DIPEA solution were dissolved in 2 ml of the DMF, and then 1.1 mmol (60.5 mg) of propargylamine was added to a resulting mixed solution; after stirring overnight at a room temperature (25° C.), a reaction solution was directly purified by HPLC to obtain the compound 1.

(2) Synthesis of a Compound 3

An excess of the compound 1 (0.2 mmol, 137.76 mg) was dissolved in 10 ml of ddH₂O, and then a mixture and a compound 2 (0.1 mmol, 37.7 mg) were added to 5 ml of the ddH₂O. A mixed solution was stirred to obtain a clear solution. 1 mL of an aqueous solution containing CuSO₄ (12.5 mg, 0.05 mmol) and sodium ascorbate (19.8 mg, 0.1 mmol) was added to initiate a Click reaction. Under a nitrogen atmosphere, a reaction solution was stirred at a room temperature (25° C.) for 24 h, and a product 3 was separated and purified by HPLC.

(3) Formation of a NO Hydrogel

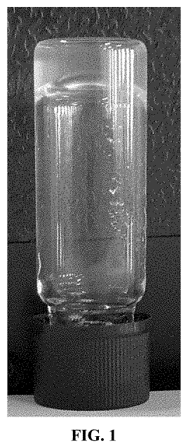

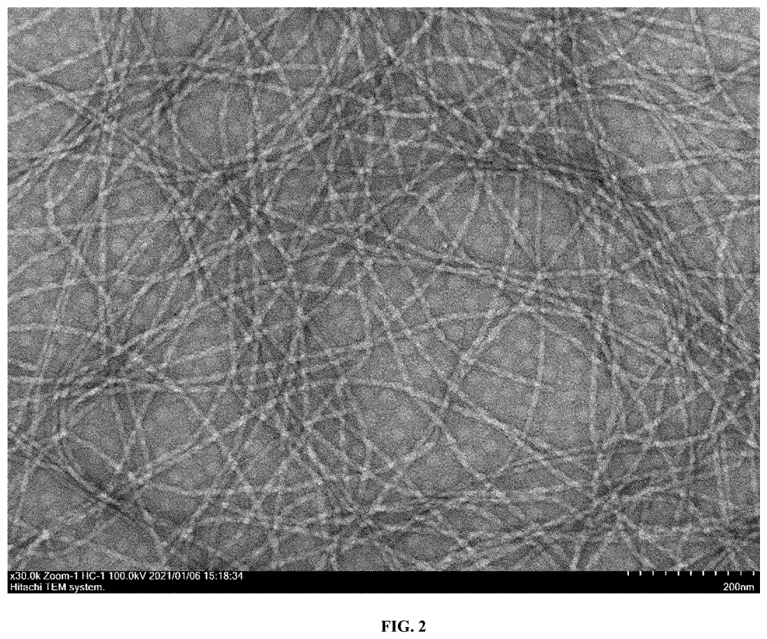

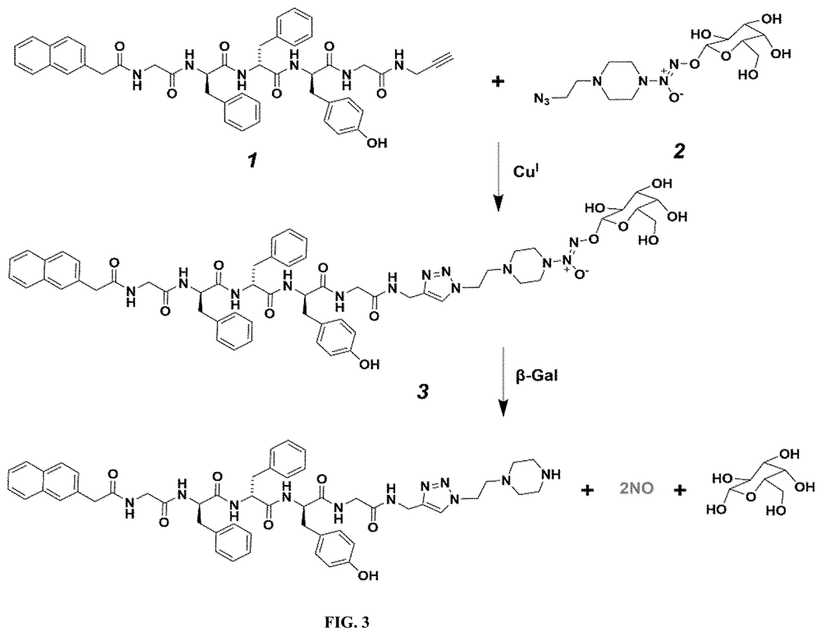

5.0 mg of a purified compound 3 was placed in a 2 mL glass bottle, added with 1 mL of a PBS solution (pH=7.4), adjusted to a pH value of 7.4 with a sodium carbonate solution, heated to boiling to completely dissolve the compound, and cooled to a room temperature to obtain a transparent and invertible hydrogel. An optical photograph of the hydrogel was shown in FIG. 1. FIG. 1 showed a vial inverted after preparation of the hydrogel at a bottom of the vial using the compound 3; the hydrogel did not flow down from the bottom of the vial after the vial was inverted, indicating that the hydrogel had a certain viscoelasticity, which was different from a fluid. The hydrogel was coated on a copper mesh and observed with a transmission electron microscope (FIG. 2). Long fibers with a diameter around 10 nm were observed, indicating that the compounds self-assembled to form an ordered nanostructure.

(4) NO Hydrogel Releasing NO Under Enzyme Catalysis

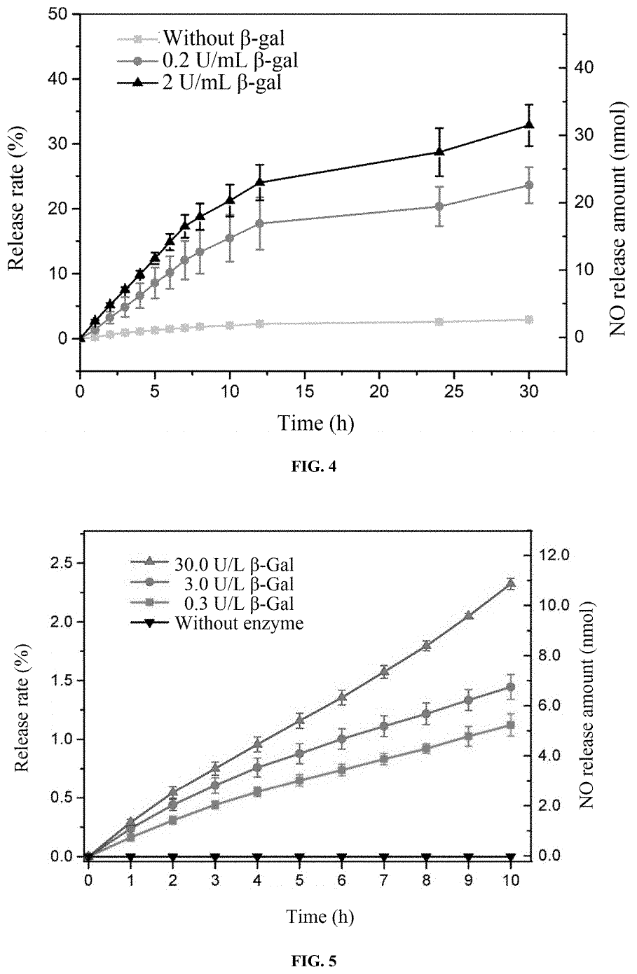

50 μL of mouse plasma containing various concentrations of β-gal (0, 0.2, and 2 U/mL), or 50 μL of PBS containing various concentrations of the β-gal (0, 0.3, 3, and 30 U/L) was added on a top of 50 μL of the hydrogel (the compound 3 was in PBS and had a concentration of 1 mg/mL). The NO released by hydrogel was measured by a Griess kit at the time points shown in FIG. 4 and FIG. 5. As shown in FIG. 4, the hydrogel did not release NO in the serum without enzyme, released 22 nmol of NO in total in the serum containing 0.2 U/mL of β-Gal within 30 h, and released 32 nmol of NO in total in the serum containing 2 U/mL of β-Gal within 30 h. As shown in FIG. 5, the hydrogel did not release NO in the PBS without enzyme, and released 5 nmol, 7 nmol, and 11 nmol of NO in total in the PBS containing 0.3 U/mL, 3 U/mL, and 30 U/mL of β-Gal within 10 h.

(5) NO Hydrogel+Enzyme Improving Sensitivity of Tumor Cells to Radiotherapy Under Hypoxia

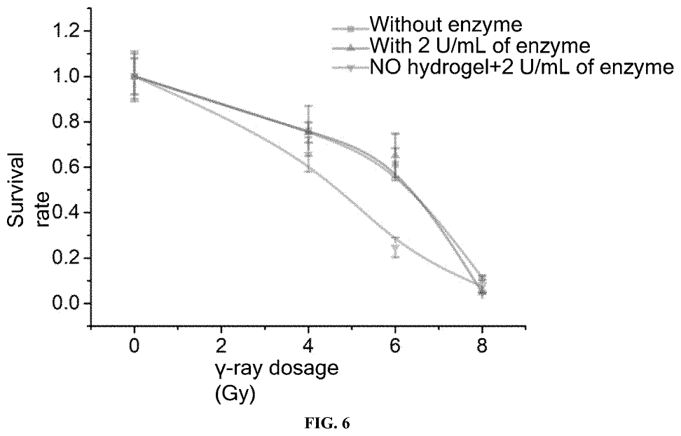

The melanoma cell line B16 cells were cultured under hypoxia for 24 h, after which a normal medium, a medium containing 2 U/mL of β-gal, and a medium containing 1 mg/mL of hydrogel and 2 U/mL of β-gal were added separately to treat for 24 h, followed by γ-ray radiation. As shown in FIG. 6, the survival rate of tumor cells in the non-enzyme group and the enzyme-added group were the same under irradiation at dosages of 2 Gy, 4 Gy, and 6 Gy, indicating that the β-gal had no toxicity to B16 tumor cells. After the tumor cells were treated with hydrogel+enzyme, the survival rates each were lower than that of the untreated group under radiation irradiation at dosages of 2 Gy, 4 Gy, and 6 Gy, indicating that the release of NO from hydrogel+enzyme improved the lethality of radiotherapy to tumor cells.

(6) NO Hydrogel Promoting Tumor Radiosensitization

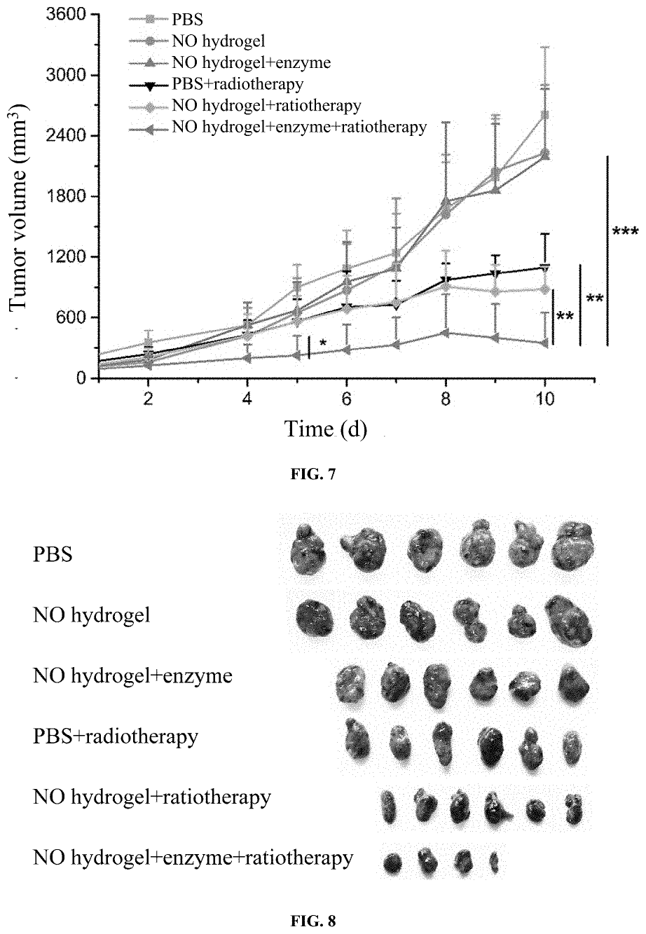

Subcutaneous injection of B16 tumor cells was conducted in C57 mice; when a volume of the B16 tumor reached about 100 mm³, the tumor-bearing mice were divided into 6 groups: a PBS group, a NO hydrogel group, a NO hydrogel+enzyme group, a PBS+radiotherapy group, a NO hydrogel+radiotherapy group, and a NO hydrogel+enzyme+radiotherapy group. For the NO hydrogel and NO hydrogel+radiotherapy groups, 50 μL of the NO hydrogel at a concentration of 5 mg/mL was injected directly into the tumor without β-Gal. For the NO hydrogel+enzyme and NO hydrogel+enzyme+radiotherapy groups, half an hour before radiotherapy, a same amount of the NO hydrogel was injected intratumorally, followed by intravenous injection of the β-Gal (a total of 4 U was dissolved in 50 μL of a PBS solution). The same treatment was repeated every 2 d for three times, reaching the end of the experiment on day 11, during which the tumor volume of the mice was measured. The results in FIG. 7 and FIG. 8 showed that treatments of the NO hydrogel group and the NO hydrogel+enzyme group could improve the sensitivity of tumors to radiotherapy and enhance an inhibitory effect of the radiotherapy on tumors; and the NO hydrogel+radiotherapy group showed the best curative effect among all groups, with the smallest mean tumor volume.

Mice were sacrificed at the end of the experiment on day 11. Tumors in each group were collected, photographed, and measured. The results showed that in the NO hydrogel+enzyme+radiotherapy group, one mouse had an extremely small tumor, and the tumors of two mice disappeared, indicating that the NO hydrogel+enzyme+radiotherapy group had the best anti-tumor effect.

(7) NO Hydrogel Promoting Vascular Normalization at Tumor Sites

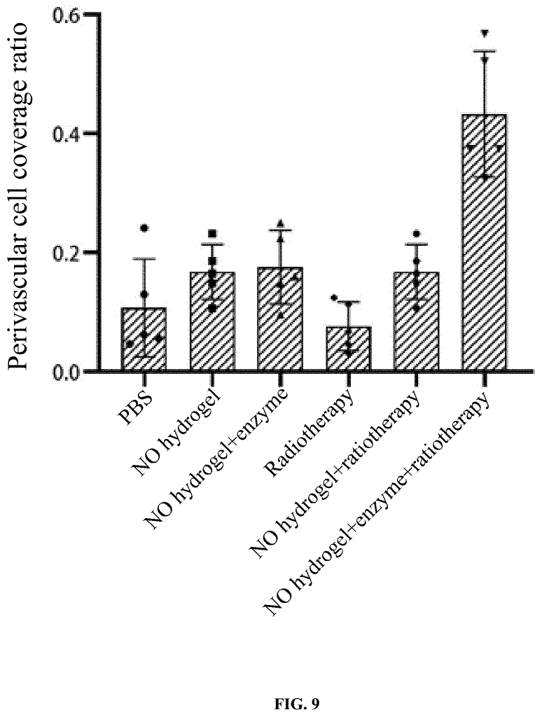

The tumor tissue was frozen, sectioned, and immunofluorescently stained, and co-stained with a vascular marker CD31 and a peripheral cell marker NG2, where CD31 was used as a normalized vascular marker at an NG2 double-positive staining site; the densities of normal blood vessels on 5 fluorescent photographs were counted and plotted (FIG. 9). FIG. 9 showed that the NO hydrogel+enzyme+radiotherapy group had the highest degree of vascular normalization. Meanwhile, the NO hydrogel group showed a higher degree of vascular normalization than the PBS group, regardless of whether the enzyme was added or not. This indicated that the NO hydrogel could promote the vascular normalization at tumor sites.

The above described are merely preferred embodiments of the present disclosure, and not intended to limit the present disclosure. Any modifications, equivalent replacements and improvements made within the spirit and principle of the present disclosure should all fall within the scope of protection of the present disclosure.

1. A nitric oxide hydrogel for promoting tumor vascular normalization and radiosensitization, comprising a gel-forming polypeptide, configured to produce a hydrogel and a β-galactose-protected nitric oxide (NO) donor molecule, wherein the gel-forming polypeptide and the β-galactose-protected NO donor molecule are covalently linked.

2-8. (canceled)

9. The nitric oxide hydrogel according to claim 1, wherein the gel-forming polypeptide is a polypeptide comprising amino acid sequences GFFY and FFG.

10. The nitric oxide hydrogel according to claim 9, wherein in the GFFY amino acid sequence, each FFY has an L-configuration or a D-configuration; and in the FFG amino acid sequence, each FF has the L-configuration or the D-configuration.

11. The nitric oxide hydrogel according to claim 1, wherein the β-galactose-protected NO donor molecule has a structural formula as follows:

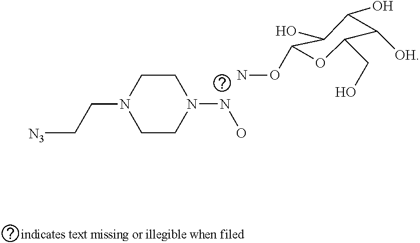

12. The nitric oxide hydrogel according to claim 1, wherein the nitric oxide hydrogel releases NO under the catalysis of β-galactosidase (β-Gal).

13. The nitric oxide hydrogel according to claim 12, wherein after intratumoral injection of the hydrogel, the β-Gal in a tumor environment continuously triggers NO release, providing local sustained release of NO at a low dosage; and
immediate intravenous injection of the β-Gal after radiotherapy is capable of releasing a large amount of NO.

14. A method of making the hydrogel according to claim 1, comprising:
S1: synthesizing the gel-forming polypeptide by polypeptide solid-phase synthesis; and
S2: linking the β-galactose-protected NO donor molecule to the gel-forming polypeptide by Click chemistry.

15. A drug for radiotherapy of cancer, comprising the nitric oxide hydrogel according to claim 1.

16. The drug for radiotherapy of cancer according to claim 15, wherein the gel-forming polypeptide is a polypeptide comprising amino acid sequences GFFY and FFG;

17. The drug for radiotherapy of cancer according to claim 16, wherein in the GFFY amino acid sequence, each FFY has an L-configuration or a D-configuration; and in the FFG amino acid sequence, each FF has the L-configuration or the D-configuration.

18. The drug for radiotherapy of cancer according to claim 15, wherein the β-galactose-protected NO donor molecule has a structural formula as follows:

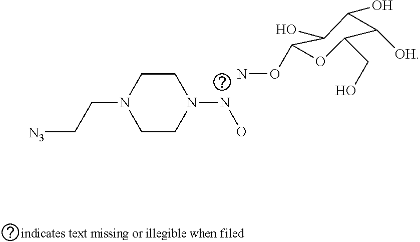

19. The drug for radiotherapy of cancer according to claim 15, wherein the hydrogel releases NO under the catalysis of β-galactosidase (β-Gal).

20. The drug for radiotherapy of cancer according to claim 19, wherein after intratumoral injection of the hydrogel, the β-Gal in a tumor environment continuously triggers NO release, providing local sustained release of NO at a low dosage; and
immediate intravenous injection of the β-Gal after radiotherapy is capable of releasing a large amount of NO.

21. The nitric oxide hydrogel according to claim 1, wherein the gel-forming polypeptide has a general formula of R-GFFY, R-GFFYG, R-GFFYGG, R-GFFYGGG, R-FF, R-FFG, R-FFGG, or R-FFGGG, and R is selected from the group consisting of H, acetic acid (Ac-), naphthylacetic acid (Nap-), and 9-fluorenylmethoxycarbonyl (Fmoc-).

22. The nitric oxide hydrogel according to claim 1, wherein the gel-forming polypeptide comprises an alkynyl located at a C-terminus of the polypeptide or on one of the amino acid side chains of the polypeptide.

23. The drug for radiotherapy of cancer according to claim 15, wherein the gel-forming polypeptide has a general formula of R-GFFY, R-GFFYG, R-GFFYGG, R-GFFYGGG, R-FF, R-FFG, R-FFGG, or R-FFGGG, and R is selected from the group consisting of H, acetic acid (Ac-), naphthylacetic acid (Nap-), and 9-fluorenylmethoxycarbonyl (Fmoc-).

24. The drug for radiotherapy of cancer according to claim 15, wherein the gel-forming polypeptide comprises an alkynyl located at a C-terminus of the polypeptide or on one of the amino acid side chains of the polypeptide.
